(19) United States
(12) Patent Application Publication
(10) Pub. No.: US 2022/0040482 A1
(43) Pub. Date: Feb. 10, 2022

(54) MODULAR IMPLANT POSITION MANIPULATOR SYSTEM

(71) Applicant: IotaMotion, Inc., Iowa City, IA (US)

(72) Inventors: Christopher Kaufmann, Iowa City, IA (US); Allan Henslee, Houston, TX (US); Adam Hahn, Pittsburgh, PA (US); Parker Reineke, North Liberty, IA (US); Marlan Hansen, Solon, IA (US)

(21) Appl. No.: 17/276,362

(22) PCT Filed: Oct. 1, 2019
(86) PCT No.: PCT/US2019/054141

§ 371 Date: Mar. 15, 2021

Related U.S. Application Data
(60) Provisional application No. 62/739,603, filed on Oct. 1, 2018.

(51) Int. Cl. A61N 1/36; A61N 1/05; A61B 34/20

(52) U.S. Cl. CPC A61N 1/36039; A61N 1/0541; A61B 2034/2051; A61B 2034/2059; A61B 34/20

(57) ABSTRACT

This document discusses, among other things, systems and methods for device-assisted manipulation of an implant's position in a patient, such as for delivering and positioning a cochlear implant. An exemplary modular system includes an implant position manipulator (IPM) unit reversibly engaging an elongate member of an implant, and a user control device for applying driving force to the IPM unit to regulate the motion of the implant. The system may include sensors providing feedback on the position or the motion of the implant, or the force or friction applied to the implant during the implantation procedure.

### CLAIM OF PRIORITY

This patent application claims the benefit of priority to U.S. Provisional Patent Application No. 62/739,603 filed Oct. 1, 2018, which is incorporated by reference herein in its entirety.

### TECHNICAL FIELD

This document relates generally to medical systems and more particularly to systems, devices, and methods for device-assisted manipulation of an implant position inside a body.

### BACKGROUND

The cochlea is the auditory portion of the inner ear. It comprises a spiraled, hollow, conical chamber of bone in which sound waves propagate from the base to the apex of the cochlea. The sound waves vibrate the perilymph that moves hair cells in the organ of Corti, converting the vibrations to electrical signals that are sent to the cochlear nerve. The hair cells and nerves in the basal or outer region of the spiraled cochlea are more sensitive to higher frequencies of sound, and are frequently the first part of the cochlea to lose sensitivity. The apical or inner region of the spiraled cochlea is more sensitive to lower frequencies.

Moderate to profound hearing loss affects a large amount of people worldwide, and may have a significant impact on a patient physical and mental health, education, employment, and overall quality of life. Hearing loss may be caused by partial damage to the cochlea. Many patients with various degrees of hearing loss have partial damage to the cochlea in the high-frequency regions (basal cochlea) from common causes such as noise exposure, drugs, genetic mutations or aging, but may retain adequate low-frequency hearing.

Cochlear implants have been used to treat patients with hearing loss. A cochlear implant is a medical device that comprises an external sound processor, a subcutaneously implantable stimulator, and an electrode assembly sized and shaped for cochlear insertion. The sound processor can convert sound signals into electrical signals, and transmit the electrical signals to the implantable stimulator. Based on the physical properties (e.g., frequencies) of the received electrical signals, the stimulator can generate electrical impulses to stimulate specific regions in the cochlea via an array of electrodes on the electrode assembly surgically inserted into the cochlea. The region for stimulation may be determined based on the frequencies of the received electrical signals. For example, higher frequencies may result in stimulation at the outer or basal cochlear region, and lower frequencies may result in stimulation at the inner or apical cochlear region.

For patients who have lost high-frequency hearing and consequently have significant difficulty with word understanding but who have substantial residual, low-frequency hearing function in apical cochlea, a short electrode assembly may be indicated to electrically stimulate the basal or outer cochlea to restore high-frequency hearing. A cochlear implant surgery may be performed by a surgeon who manually inserts the electrode assembly into the damaged portion of a patient cochlea (e.g., basal cochlea), while avoiding or minimizing any trauma to the undamaged cochlear regions to preserve the low-frequency hearing function. The cochlear implant may be used together with a hearing aid that acoustically stimulates the undamaged low-frequency sensitive apical cochlea.

Intracochlear trauma can occur from large pressure spikes generated during the insertion of cochlear implant electrodes. Cochlear implant surgery can also involve insertion of a guide sheath or tube near or partially into the cochlea. Insertion of any solid or flexible bodies, tubes, or sheaths into the cochlea could elicit similar fluid and force spikes. These pressures spikes may be of sufficient intensity to cause trauma similar to that of an acoustic blast injury and are one likely source for postoperative loss of residual hearing. Similar to the insertion trauma cause by electrode insertion, the manual insertion of a sheath or other solid body/tube into the cochlea may also cause intracochlear fluid pressure spikes and intrachochlear damage.

### SUMMARY

A hearing-preservation cochlear implant surgery involves implanting an electrode assembly into the damaged cochlear region, while avoiding any trauma to the undamaged cochlear region to preserve any normal residual hearing. In current cochlear implant surgery, complete manual insertion electrode assembly into patient cochlea may cause undesirable outcome in some patients. For example, manual insertion of electrode assembly may lack precision in implant position and motion control, such as the control of insertion rate, distance, or forces applied to the implant for advancing the electrode assembly to the target cochlear region. This may cause damage to fragile cochlear structures such as local trauma to cochlea wall and hair cells, and result in residual hearing loss.

Complete manual positioning of cochlear implants may also be subject to high inter-operator variability among surgeons. The inter-operator variability is demonstrated in dramatic differences in patient outcomes between institutions and surgeons of differing skill levels. Some patients undergoing hearing-preservation cochlear implant surgery may experience additional hearing decline weeks to years after surgery. Such a continual decline in hearing function may be attributed to an inflammatory response to the trauma inflicted during an initial cochlear implant surgery. Some clinical studies show that techniques aimed at reducing electrode-insertion forces during surgery have improved patient hearing preservation outcomes. For at least the foregoing reasons, the present inventors have recognized that there remains a need to improve surgical precision of implant delivery and positioning, and to reduce the risk of perioperative trauma to undamaged cochlea region.

This document discusses, among other things, systems, devices, and methods for device-assisted positioning of an implant in a patient, such as implantation or repositioning of a cochlear implant in a hearing-preservation cochlear implant surgery. The systems and methods discussed herein may also be adapted for controlling insertion of a guide sheath or tube that may be used in conjunction with electrode implantation. An exemplary modular system includes an implant position manipulator (IPM) unit reversibly interfacing with and securely engaging an implant, such as a cochlear implant having an elongate member, and a user control device for providing force and motion to the IPM unit to regulate the motion of the implant. The system may include sensors providing feedback on the position or the motion of the implant, or the force or friction applied to the implant during the implantation procedure. The control systems may also interface with external systems providing electrophysiological measures to enable closed loop feedback on electrode positioning in real-time during implantation.

Example 1 is a system for positioning an implant in a patient. The system comprises: an implant position manipulator (IPM) unit, including a coupling unit configured to reversibly engage an elongate member of the implant and to deliver and position the implant into a target implantation site; and a user control device operably coupled to the IPM unit, the user control device configured to apply driving force to the IPM unit to control delivery and positioning of the implant into the target implantation site.

In Example 2, the subject matter of Example 1 optionally includes a cochlear implant having an electrode array disposed on a portion of the elongate member.

In Example 3, the subject matter of any one or more of Examples 1-2 optionally includes the user control device that can be operatively coupled to the IPM unit via a mechanical coupling including an engagement unit of the user control device and an engagement unit of the IPM unit. The engagement unit of the user control device matches in size and shape of the engagement unit of the IPM unit.

In Example 4, the subject matter of any one or more of Examples 1-3 optionally includes the user control device that can be operatively coupled to the IPM unit via a magnetic or electromagnetic coupling between a magnetic or electromagnetic coupler of the user control device and an magnetic or electromagnetic coupler of the IPM unit.

In Example 5, the subject matter of any one or more of Examples 1-4 optionally includes the user control device which is a handheld device.

In Example 6, the subject matter of any one or more of Examples 1-5 optionally includes the user control device that can be an unpowered device including a handle for a user to directly apply the driving force.

In Example 7, the subject matter of any one or more of Examples 1-5 optionally includes the user control device that can be a powered device including an electric motor configured to produce the driving force to control the IPM unit.

In Example 8, the subject matter of Example 7 optionally includes the powered device that can include control mechanisms to adjust the driving force or a motion of the user control device.

In Example 9, the subject matter of any one or more of Examples 1-8 optionally includes the user control device that can include an output unit configured to present one or more motion parameters of the implant.

In Example 10, the subject matter of any one or more of Examples 1-9 optionally includes a sensor configured to sense one or more motion parameters of the implant.

In Example 11, the subject matter of Example 10 optionally includes a Hall-effect sensor configured to sense a position or a displacement of the elongate member of the implant inside the patient.

In Example 12, the subject matter of Example 10 optionally includes a force sensor configured to sense an indication of force or friction imposed on the elongate member of the implant during implantation.

In Example 13, the subject matter of any one or more of Examples 10-12 optionally includes the sensor that can be included in the IPM unit.

In Example 14, the subject matter of any one or more of Examples 10-12 optionally includes the sensor that can be included in the user control device.

In Example 15, the subject matter of any one or more of Examples 1-14 optionally includes a physiologic sensor configured to sense one or more electrophysiological signals.

In Example 16, the subject matter of any one or more of Examples 1-15 optionally includes the coupling unit that can include at least first and second rollers arranged and configured to engage, through compression between respective radial outer surfaces of the first and second rollers, a portion of the elongate member of the implant. The user control device can be configured to apply the driving force to the first roller to cause rotation thereof that propels the implant through friction generated by the compression.

In Example 17, the subject matter of Example 16 optionally includes the IPM unit that can further include a transmission unit configured to transmit the driving force to the first roller.

In Example 18, the subject matter of Example 17 optionally includes transmission unit that can include a gear-reduction device configured to alter motion rate or direction produced by the driving force.

In Example 19, the subject matter of Example 18 optionally includes the gear-reduction device that can include a gear train of spur gear arrangement.

In Example 20, the subject matter of Example 18 optionally include, wherein the gear-reduction device that can include a gear train of worm-gear arrangement.

In Example 21, the subject matter of Example 16 optionally includes the IPM unit that can include at least one slide spring-biased against the first roller via a compression spring, and that the second roller is spring loaded and configured to be attached to the at least one slide and to engage at least a portion of the elongate member of the implant through compression between the respective radial outer surfaces of the first and second rollers.

In Example 22, the subject matter of any one or more of Examples 1-15 optionally includes the coupling unit that can include a drive roller and an implant carrier. The implant carrier can be configured to detachably hold at least a portion of the elongate member of the implant; and the user control device is configured to apply the driving force to the drive roller to cause rotation thereof that propels the implant carrier along with the elongate member.

In Example 23, the subject matter of Example 22 optionally includes the implant carrier that can be coupled to a drive roller via a transmission screw. The transmission screw can be configured to convert a rotary motion of the drive roller to a linear motion of the implant carrier.

In Example 24, the subject matter of Example 22 optionally includes the transmission screw that can include an Archimedes' screw.

In Example 25, the subject matter of Example 22 optionally includes the transmission screw that can include a worm.

In Example 26, the subject matter of any one or more of Examples 1-25 optionally includes a housing to enclose the IPM unit, the housing including a sealable opening for the user control device to access the IPM unit.

In Example 27, the subject matter of Example 26 optionally includes the housing that can include a viewing window to display an indication of implant position.

In Example 28, the subject matter of Example 27 optionally includes the indication of implant position that can include markers on the coupling unit.

In Example 29, the subject matter of Example 27 optionally includes the indication of implant position that can include graduations on an exterior of the housing.

In Example 30, the subject matter of any one or more of Examples 1-29 optionally includes the IPM unit that can further include a fixation member configured to detachably affix the IPM unit to the patient.

In Example 31, the subject matter of any one or more of Examples 1-30 optionally includes an IPM unit configured for subcutaneous implantation, and the user control device can be configured for transcutaneous coupling to the IPM unit.

In Example 32, the subject matter of any one or more of Examples 1-31 optionally includes a sheath extended from the IPM unit to a surgical entrance of the target implantation site, the sheath configured to at least partially enclose the elongate member.

In Example 33, the subject matter of Example 32 optionally includes sheath bellows within the sheath. The sheath bellows can be configured to extendably seal the elongate member as the implant advances into the target implantation site.

In Example 34, the subject matter of Example 32 optionally includes the sheath that can include a drug-eluting sheath.

In Example 35, the subject matter of any one or more of Examples 32-34 optionally includes the IPM unit that can include a drug reservoir to store pharmacological agents controllably released to the sheath.

Example 36 is a method for delivering and positioning an implant with an elongate member into a target implantation site of a patient via a device-assisted implant positioning system. The method comprises steps of: engaging at least a portion of the elongate member of the implant to an implant position manipulator (IPM) unit of the implant positioning system; affixing the IPM unit to the patient via a fixation member of the IPM unit; engaging a user control device to the IPM unit; and applying driving force to the IPM unit to control delivery and positioning of the implant into the target implantation site.

In Example 37, the subject matter of Example 36 optionally includes the engagement of the elongate member that can include engaging at least a portion of the elongate member of the implant through compression between respective radial outer surfaces of first and second rollers; and wherein applying the driving force includes driving rotation of the first roller that propels the implant through friction generated by the compression between the radial outer surfaces of the first and second rollers.

In Example 38, the subject matter of Example 36 optionally includes the engagement of the elongate member that can include attaching at least a portion of the elongate member of the implant to an implant carrier coupled to a drive roller via a transmission screw. The application of the driving force can include driving rotation of the drive roller that propels the implant carrier along with the elongate member.

In Example 39, the subject matter of any one or more of Examples 36-38 optionally includes enclosing the at least a portion of the elongate member of the implant within a sheath or sheath bellows.

In Example 40, the subject matter of any one or more of Examples 36-39 optionally includes applying the driving force through an electric motor within, or connected to, the user control device.

In Example 41, the subject matter of any one or more of Examples 36-40 optionally includes steps of: sensing one or more motion parameters of the elongate member or electrophysiological signals during the implantation of the implant; and controlling the IPM unit to propel the implant according to the sensed one or more motion parameters.

The systems, devices, and methods discussed in this document may improve the technology of device-assisted implantation and positioning of an implant or prosthesis.

Compared to complete manual insertion and implant steering, using the IPM unit and the user control device as described herein can help achieve more precise force and movement control of the implant in a hearing-preservation cochlear implant surgery, reduce mechanical forces imposed on the delicate cochlear structure such as basilar membrane and organ of Corti and force variations, and thus minimize the risk of trauma on the undamaged structure at the apical cochlea. This may ultimately better preserve patient residual natural hearing. With more consistency of implant motion and less intra-operator and inter-operator variability, the systems and methods discussed herein allow more people with disabling hearing loss to hear better over their lifetimes.

Some implantation positioning systems include a power unit (e.g., batteries or transcutaneous power transfer means) and electronic control circuitry to robotically control the implant delivery and positioning. The power unit and the electronics can take more space of the implant system, such that the implant positioning unit may be too bulky for subcutaneous implantation. The inclusion of the power unit and the electronics also increases system complexity and cost. In this document, a device-assisted IPM unit eliminates the internal power supply. Under direct control from a user (e.g., a surgeon), the IPM unit may be accessed externally, or via a minimally invasive or subcutaneous access point to modify the position of the implant. Compared to the powered, robotically controlled implantation system, the present system is more compact and lighter, which makes it suitable for external fixation or internal implantation. Additionally, it has reduced system complexity and lower cost, improved user experience with more direct feedback, yet can still provide fine precision of implant position control.

The modular design of the present IPM system allows for easy replacement or interchange of a particular module. This may not only improve the system reusability and efficiency, but may also reduce the cost of system maintenance. For example, the IPM unit may be disposable, single-use device positioned in a sterile surgical field or in contact with the patient during an implantation surgery. Alternatively, the IPM unit may be subcutaneously implanted. The user control device may be a reusable device that matches in shape and configuration with the IPM unit, and can engage with interchangeable IPM unit.

The present IPM system can provide better patient care at a lower cost by addressing some critical unmet needs. The present technology may enhance ability to preserve residual hearing by limiting insertional forces and trauma. It offers the ability to dynamically adjust the depth of an electrode array in-office to best fit a patient's evolving hearing loss over a lifetime with no further surgery. For example, advancement of the implant electrode may be customized with no implant removal and re-implantation with a full-length electrode. Insertion depth may be customized according to each patient's hearing demands and may be adjusted dynamically according to the patient's evolving hearing needs. In addition, by controlling implantation rate and forces, the rate of advancement and forces to advance the electrode array may be optimized to minimize trauma, and enhance preservation of residual cochlear structures and hearing. This will reduce overall healthcare costs, significantly enhance the outcomes achieved with current electrode systems, and may significantly expand the cochlear implant candidacy range, adding value for patients, healthcare systems, and implant companies.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 is a diagram illustrating, by way of example and not limitation, a device-assisted implant positioning system and portions of an environment in which the system may operate.

FIGS. 2A-2B are diagrams illustrating, by way of example and not limitation, IPM units coupled to the elongate member of an implant, such as a cochlear implant.

FIGS. 3A-3B are diagrams illustrating an embodiment of the IPM unit shown in FIG. 2A.

FIGS. 4A-4B are diagrams illustrating another embodiment of the IPM unit shown in FIG. 2A.

FIGS. 5A-5B are diagrams illustrating another embodiment of the IPM unit shown in FIG. 2A.

FIG. 6A-6B illustrate, by way of example and not limitation, user control devices that may be coupled to the IPM unit to activate the IPM unit to move an implant.

FIGS. 7A-7B are cutaway diagrams illustrating, by way of example and not limitation, a sheath and optional sheath bellows that enclose at least a portion of the elongate member of the implant.

FIG. 8 is a flowchart illustrating, by way of example and not limitation, a method of manipulating an implant's position in a patient via an external or subcutaneously implantable system.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Disclosed herein are systems, devices, and methods for device-assisted implantation, positioning, and manipulation of an implant in a patient, such as insertion of a cochlear implant and/or guide sheath during a hearing-preservation cochlear implant surgery. The system can include an implant position manipulator (IPM) unit to engage an implant, and a user control device operatively coupled to the IPM unit. An operator (e.g., a surgeon) can use the user control device to directly operate the IPM unit, control with sufficient precision the implant movement and position, and deliver the implant to a target implantation site. The systems, devices, and methods discussed herein improves implant positioning precision and consistency based on the patient's evolving needs without repeated surgical interventions. The technology allows a full-length cochlear implant electrode to be partially inserted for preservation of patient residual hearing and cochlear structures. In the case that patient hearing continues to decline over time, a clinician can extend an original full length of the implant.

Although the discussion in this document focuses on cochlear implant, this is meant only by way of example and not limitation. It is within the contemplation of the present inventors, and within the scope of this document, that the systems, devices, and methods discussed herein may be configured for device-assisted delivering, positioning, steering, or extracting various types of implants or prosthesis as well as associated instruments. By way of non-limiting examples, the implants may include leads, catheter, guidewire, guide sheath, neuroprosthesis, implantable stimulator such as spinal cord stimulator, brain stimulator, or other tubular implanted medical devices. The implants may be designed for temporary or permanent implantation. The implants may be used for medical diagnosis of a disease or other conditions such as diagnostic catheters, or for therapeutic purposes of cure, mitigation, treatment, or prevention of disease, such as implantable electrodes for stimulating cardiac, neural, muscular, or other tissues. In addition to new implantation, the systems, devices, and methods discussed herein may also be used to surgically reposition or replace an existing implant.

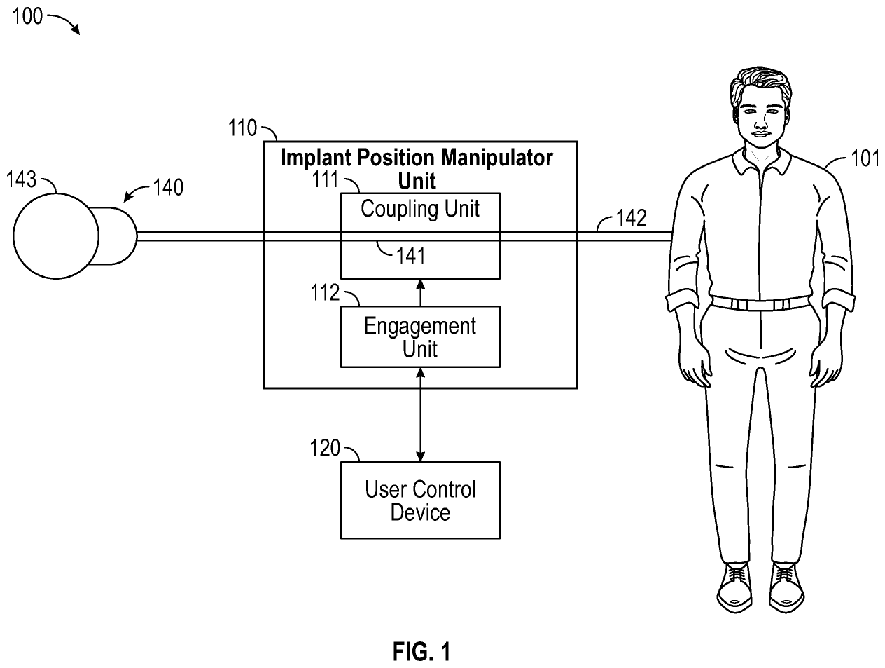

FIG. 1 illustrates a device-assisted implantation system 100 and portions of an environment in which the system 100 may operate. The device-assisted implantation system 100 may include an IPM unit 110 and a user control device 120. The device-assisted implantation system 100 may engage, deliver, and position an implant 140 into a target implantation site of a patient 101.

The implant 140 may include an elongate member 141. The elongate member 141 may be an integral part of the implant 141, such as a tubular implant body or an elongate shaft. Examples of such an implant may include an implantable lead or catheter. Alternatively, the elongate member 141 may be a part of a delivery system detachably coupled to the implant. Examples of such an implant may include a guidewire or an introducer that may snatch an implant at a particular location, such as at a distal portion of the elongate member 141. The IPM unit 110 may move the elongate member 141, thereby transporting the implant to a target implantation site. Once the implant has reached and been securely positioned at the target implantation site, the elongate member 141 may be disengaged from the implant, and the IPM unit 110 may retract the elongate member 141 away from the patient 101.

By way of example and not limitation, the implant 140 may include a cochlear implant for treating hearing loss through electrostimulation of a specified cochlea region. The cochlear implant, as illustrated in FIG. 1, may include an implantable stimulator 143 and the elongate member 141 with an electrode array such as disposed at distal portion 142 of the elongate member 141. The implantable stimulator 143, which may be implanted under the scalp, can generate electrical impulses, and deliver the electrical impulses to the electrode array through conductors in the elongate member 141. The electrode array may be surgically inserted into and positioned at the target cochlear site. In patients with impaired high-frequency hearing function but preserved low-frequency hearing function, a short electrode array of the implant may be positioned at the outer or basal cochlear region to deliver electrostimulation therein to restore high-frequency hearing function.

In some examples, the implant 140 may be delivered through a guide sheath, which is alternatively or additionally controlled by the IPM unit 110. In some examples, the IPM unit 110 includes separate structures to control a guide sheath separately from the implant 140. In other examples, the guide sheath may be positioned initially by the IPM unit 110, and the implant 140 implanted through the previously positioned guide sheath. In this example, the implant 140 may be controlled by the IPM unit 140 once the guide sheath is in place.

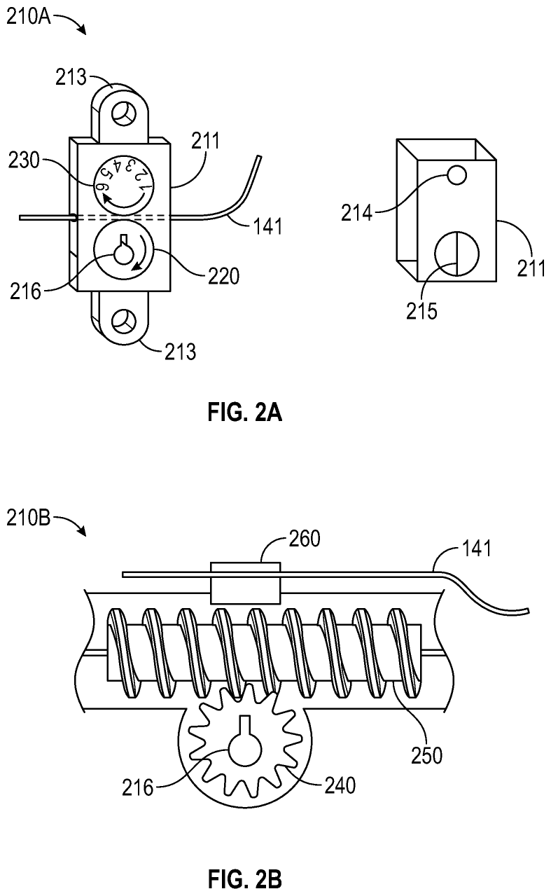

The IPM unit 110 may be a non-implanted external device, or a subcutaneously implantable device. The IPM unit 110 includes a coupling unit 111 and an engagement unit 112. The coupling unit 111 is configured to interface with the elongate member 141 of the implant, and frictionally move the elongate member 141 in a specific direction (e.g., forward for implant insertion, or reverse for implant extraction), at a specific rate, or for a specific distance relative to a reference point such as the interface between the coupling unit 111 and the elongate member 141. Examples of the coupling unit 111 may include a leadscrew, a clamp, a set of rotors, or a rack and pinion arrangement, among other coupling mechanisms. In an example, the coupling unit 111 can compress against at least a portion of the elongate member 141 to produce sufficient friction between the coupling unit 111 and the elongate member 141. In another example, the coupling unit 111 can include a movable object detachably attached to the elongate member 141. When attached, the movable object can hold the elongate member 141 to move linearly under user control, thereby transporting the associated implant to a target implantation site. Examples of coupling unit 111 are discussed below, such as with reference to FIGS. 2A-2B.

The coupling unit 111 may include adjustable couplers for reversible or interchangeable connection between the IPM unit 110 and the elongate member 141. In the event of implant exchange or replacement, the coupling unit 111 may be adjusted to release the compression on, or the attachment to, the elongate member 141 of an existing implant, which may be then removed from the IPM unit 110. A new implant with an elongate member may be reloaded and engaged with the IPM unit 110. The IPM unit 110 need not be removed and may remain in place during implant replacement.

The engagement unit 112 serves as an access point where a user can directly access and manipulate the IPM unit 110 via a user control device 120. The engagement unit 112 may be coupled directly or indirectly to the coupling unit 111. The engagement unit 112 may be positioned subcutaneously under the skin or other minimally invasive site and enables controlled movement of an implant deep to tissue, bone, or other delicate organ structures. Subcutaneous placement of the engagement unit 112 (or the entirety of the IPM unit 110) decreases risk of infection by isolating from external environment. The engagement unit 112 may include identifiers to facilitate recognition and relocation at a future point in time when the implant needs to repositioned and the IPM unit 110 needs to be re-accessed subcutaneously. In an example, the engagement unit 112 may be located physically via distinct, palpable surface physical feature such as ridge, protrusion, or curvature from the surrounding structure. In another example, the engagement unit 112 may have radiopaque or imaging signal attenuating marker to locate via ultrasound, X-ray, or other biological imaging modality. In yet another example, the engagement unit 112 may be located via magnetic or electromagnetic signal coupled to an external magnet or signal strength detector.

The IPM unit 110 may be stably attached to a body part of the patient, or to an object at patient immediate environment such as the surgical table. The IPM unit 110 may be a compact, lightweight micromechanical device suitable for direct attachment to the patient, such as on the patient head during a cochlear implant surgery, while maintaining sufficient stability during the implantation. The IPM unit 110 may be sized and shaped to facilitate patient attachment. In an example, the IPM unit 110 may have a curved exterior contact surface that conforms to the contour of a body part of the patient 101, such as a head region. In an example, the IPM unit 110 may include a fixation member that allows for detachable affixation of the IPM unit 110 to the patient 101. The fixation may be invasive fixation that involves incision of the skin and penetration of subcutaneous tissue. Examples of the fixation member may include one or more of a screw, a pin, a nail, a wire, a hook, a suture, or a magnet within the IPM unit 110 coupled to one or more magnetic screws or pins affixed to the body part of the patient 101. In an example, the fixation member may include one or more of self-drilling screws, self-tapping screws, or self-piercing screws, such that no pilot hole needs to be drilled at the affixation site prior to screw installation. Alternatively, the fixation may be non-invasive fixation with the use of non-invasive clamps or holding devices that prevent movement relative to the patient 101.

The contact surface of the IPM unit 110 may be processed to improve stability during the implant advancement procedure. In an example, the IPM unit 110 may have an exterior surface with a rough finish, such as ridges, corrugates, teeth, or other coarse surface textures. Additionally or alternatively, the IPM unit 110 may have one or more gripping elements configured to frictionally bond the IPM unit 110 to a body part of the patient 101. The gripping elements may be distributed on a portion of the exterior surface. Examples of the gripping elements may include penetrators such as spikes, pins, or barbs protruding from the exterior surface. When the IPM unit 110 is pressed and held against the attachment region (e.g., patient head), the rough surface or the gripping elements may provide sufficient friction or gripping force to hold the IPM unit 110 in place relative to the patient 101 during the implantation advancement.

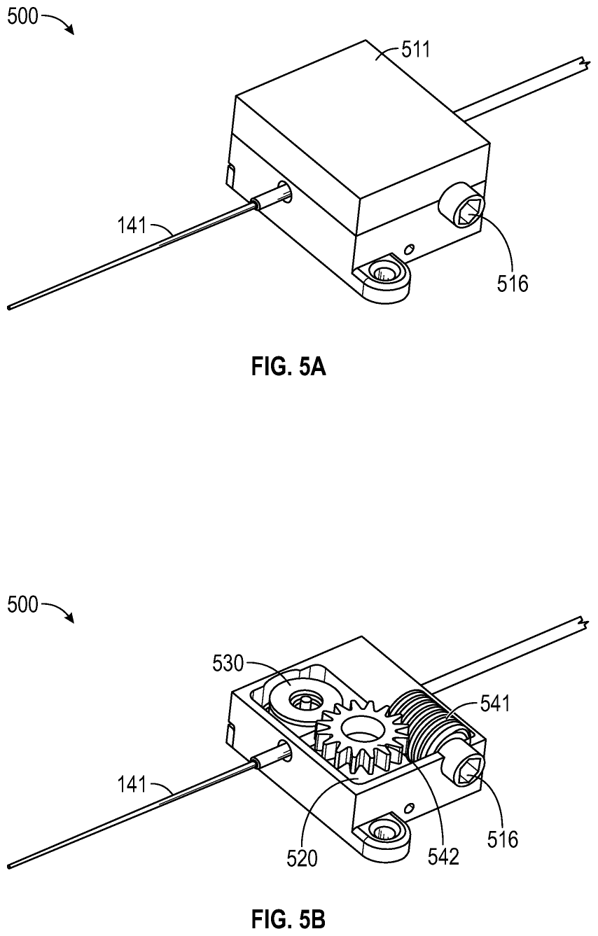

The user control device 120 may be a mechanical, electromechanical, magnetic, or electromagnetic device. When engaged with the IPM unit 110 via the engagement unit 112, the user control device 120 can cause the coupling unit 111, directly or indirectly (e.g., via a transmission unit), to move the elongate member 141 at specific rate or direction, or for a specific distance, thereby delivering and positioning the implant 140 into a target implantation site. The user control device 120 may receive information about the force, location, speed, or other parameters of the implant. Such information may be sensed by sensors disposed on the IPM unit 110, or by sensors situated within the user control device 120, as to be discussed in the following. The user control device 120 may also receive measurement data related to implant position from an external system. In some examples, the user control device 120 may receive electrophysiological information in real-time, such as electrocochleography (ECoG), neural response telemetry, cochlear response telemetry, or auditory brainstem responses (ABRs) recordings from either the cochlear implant or other ECoG recording system or method. The user control device 120 may include a display to display the motion and/or electrophysiological information, or to present visual or audio alert feedback. A user may adjust the force and/or motion output according to the motion and/or electrophysiological information or in response to the alert notification. For example, if decreases or significant changes are recorded in ECoG measures, the user control device 120 can provide immediate feedback to the surgeon in the form of visual or audible notification to alert the use to withhold further implant motion. Upon such notification, the surgeon may adjust implant insertion trajectory or system motion parameters as needed to avoid intracochlear damage or suboptimal electrode position or choose to the override notifications via physical acknowledgement mechanism. After acknowledgment of warning notification, the alert is removed and the user may continue the device-assisted insertion of the implant. Examples of the user control device are discussed below, such as with reference to FIGS. 5A-5B.

Portions of the user control device 120 may be implemented using hardware, software, firmware, or combinations thereof. Portions of the user control device 120 may be implemented using an application-specific circuit that may be constructed or configured to perform one or more particular functions, or may be implemented using a general-purpose circuit that may be programmed or otherwise configured to perform one or more particular functions. Such a general-purpose circuit may include a microprocessor or a portion thereof, a microcontroller or a portion thereof, or a programmable logic circuit, or a portion thereof. For example, a “comparator” may include, among other things, an electronic circuit comparator that may be constructed to perform the specific function of a comparison between two signals or the comparator may be implemented as a portion of a general-purpose circuit that may be driven by a code instructing a portion of the general-purpose circuit to perform a comparison between the two signals.

FIGS. 2A-2B are diagrams illustrating examples of IPM units 210A and 210B each configured to reversibly engage the elongate member 141 of an implant, such as a cochlear implant. The IPM units 210A and 210B are embodiments of the IPM unit 110 as illustrated in FIG. 1. As illustrated in FIG. 2A, the IPM unit 210A includes a housing 211 that encloses components that engage the elongate member 141 and deliver and position the implant into a implantation site. The housing 211 includes an entrance port and an exit port to let the elongate member 310 pass through the IPM unit 210A. The exterior of the housing 211 may include fixation means, such as flanges 213 with holes to receive screws or other fasteners to affix the IPM unit 110 to a body part of the patient or an object in the sterile field of surgery. Alternative fixations means may be used, such as a pin, a nail, a wire, a hook, a suture, or a magnet within the IPM unit 110 coupled to one or more magnetic screws or pins affixed to the body part of the patient.

The IPM unit 210A may include at least two rollers, a drive wheel 220 and an idler wheel 230, which are embodiments of the coupling unit 111. The drive wheel 220 and the idler wheel 230 are arranged and configured to engage at least a portion of the elongate member 141. The engagement of the elongate member 141 may be through compression between respective radial outer surfaces of the drive wheel 220 and an idler wheel 230. The drive wheel 220 may be coupled via a bearing to an axle that is securely attached to the housing 211, such that the drive wheel 220 may rotate on the axle without lateral movement relative to the housing 211.

The drive wheel 220 may be coupled to an engagement unit 216, which is an embodiment of the engagement unit 112, and sized and shaped to match an engagement unit of the user control device 120. In an example, the engagement unit 216 includes a recessed portion adapted to receive a protruding portion of the engagement unit of the user control device. In another example, the engagement unit 216 includes a protruding portion adapted to fit into a recessed portion of the engagement unit of the user control device. In an example, the engagement unit 216 is mounted on a cylindrical base coaxially aligned with and affixed to the drive wheel 200, such that when force is applied (e.g., via the user control device 120) to rotate the engagement unit 216, the drive wheel 220 can also be rotated coaxially. In another example, the engagement unit 216 may be a concentric insertion hole drilled on the drive wheel 200. The hole has a size and geometry that match the engagement unit of the user control device 120. In some examples, the engagement unit 216 may include a releasable locking mechanism (e.g., a latch) to prevent inadvertent disengagement of the coupling between the user control device and the drive wheel 220.

The idler wheel 230 may be coupled to a biasing system that includes a spring bias. Under the spring tension, the idler wheel 230 compresses against the drive wheel 220 to generate adequate friction on the elongate member 141 between the drive wheel 220 and the idler wheel 230. Rotation of the drive wheel 220 can frictionally activates rotation of the idler wheel 230, thereby propelling the elongate member 141 to a specific direction (e.g., forward or backward) at a specific rate. Because the idler wheel 230 is held in place by the biasing system rather than being affixed to the housing 211, the idler wheel 230 may move laterally relative to the housing 211. This may allow for accommodating implants with elongate members of a range of diameters or cross-sectional shapes, while maintaining sufficient friction on the elongate member for desirable movement. In an example, a user may manually move the idler wheel 230 away from the drive wheel 220, thereby release the compression and open the space between the drive wheel 220 and the idler wheel 230. The user may remove the elongate member 141 from the IPM unit 210A, or load another implant with an elongate member into the IPM unit 210A.

The elongate member 141 can have a cylindrical shape or otherwise have a convex cross-sectional profile. In some examples, the radial outer surfaces of the drive wheel 220 and the idler wheel 230 may each have a radially concave profile to allow for secure engagement of the elongate member 141. The concavity of the concave profile, which quantifies a degree of the concave surface, may be determined based on the geometry such as the diameter of the elongate member 141.

The radial outer surfaces of the drive wheel 220 and/or of the idler wheel 230 may be coated with a frictions material, such as a layer of silicone rubber, polymer, or other composite materials. Additionally or alternatively, said radial outer surface may be mechanically textured to have a rough and corrugated surface. The frictious material layer or the corrugated surface finish may increase the friction and prevent the elongate member 141 from slipping on the wheels.

The IPM unit 210A may include on the housing 211 a sealable opening that allows an easy access to the engagement unit 216 from the outside of the IPM unit 210A. A seal 215 may be used to protect the engagement unit 216 and other internal components (e.g., the wheels 220 and 230) from contamination. During operation, the seal 215 may be removed to allow the user control device 120 to access the engagement unit 216. After the operation, the seal 215 may be plugged back to the sealing position. The seal 215 may also serve as a landmark to locate the engagement unit 216 when the IPM unit 210A is subcutaneously implanted.

The IPM unit 210A may include on the housing 211 a viewing window 214 to display implant status such as an indication of current implant position. In an example as illustrated in FIG. 2A, markers may be imprinted circumferentially on the idler wheel 230 to indicate the amount of rotation of the idler wheel 230 relative to a reference point. In an example, the markers include numerical values (e.g., numbers 1 through 6) calibrated using measured distance or position of the implant. The viewing window 214 may be sized and positioned to allow a user to read a marker when the idler wheel rotates. As the amount of rotation of the idler wheel 230 represents implant advancement distance, the marker as seen from the viewing window 214 provides the user with information about current implant position.

FIG. 2B illustrates an IPM unit 210B that includes a drive wheel 240, a transmission screw 250, and an implant carrier 260. The drive wheel 240, the transmission screw 250, and at least a portion of the implant carrier 260 may be enclosed in a housing configured to affix to a body part of the patient or an object in the sterile field of surgery. A user control device may be operably engaged with the drive wheel 240 via an engagement unit 216, and drive rotation of the drive wheel 240, as similarly discussed above with reference to FIG. 2A. The drive wheel 240 may include a gear portion coupled to the transmission screw 250. Examples of the transmission screw 250 may include a worm, or an Archimedes' screw. In an example, the drive wheel 240 is coaxially coupled to a worm gear that meshes with the transmission screw 250. In another example, the drive wheel 240 is a toothed wheel with angled and curved teeth that mesh with the transmission screw 250.

The implant carrier 260 can have a threaded side with threads interfacing with the transmission screw 250, and another side including a coupler to detachably hold at least a portion of the elongate member 141. When the drive wheel 240 rotates against the transmission screw 250, the transmission screw 250 rotates, pushing the implant carrier to move linearly in line with the axel of the transmission screw 250. The transmission screw 250 can therefore convert the rotary motion of the drive wheel 240 to linear motion of the implant carrier, thereby moving the elongate member 141 of the implant forward or backward. In an example, the transmission screw 250 has a length equal to or greater than the pre-determined maximal implant movement distance, such as approximately 30-35 millimeters in a hearing-preservation cochlear implant surgery. This allows the implant carrier 260 to carry the implant to travel for the maximally allowable distance along the transmission screw 250. In an example, to provide quick and real-time feedback to the user on current implant position, the IPM unit 210B may include on the housing a viewing window to display an indication of a position of the implant carrier 260. In some examples, graduations may be marked on the housing around the viewing window to show how much implant advancement or retreatment would occur for a given user rotation.

In an example, at least a portion of the elongate member 141 may be situated within a compliant and extendable sheath compressed and stored within a compartment in the housing 211 of the IPM unit 210A or 210B. The wheels 220 and 230 can frictionally advance the elongate member 141 thereby extending it from the compartment, or frictionally retract the elongate member 141 thereby compressing it into the compartment. In some examples, a drug reservoir may be included within the housing 211 that stores pharmacological agents such as antibacterial, antifouling, antiproliferative, or antifibrotic drugs. Pharmacological agents may be controllably released during the implant advancement or repositioning, or at specified post-surgical time periods or frequency.

The IPM units 210A and 210B may include one or more sensors configured to sense position or motion of the implant, or force or friction applied to the of the implant during implantation. The sensors may be attached to one or both of the drive wheel 220 or the idler wheel 230. Examples of the sensors may include linear or rotary encoders, ferromagnetic Hall effect sensors, optical sensors, capacitive sensors configured to detect implant motion and/or position, or force sensors configured to sense indications of force or friction imposed on the implant during the implant advancement, such as axial, lateral, or radial insertion force as the implant advances into the cochlea. Sensor data may be communicated to a receiver, such as implemented in the user control device 120, via a wired connection or a wireless network, such as near-field communication (NFC) protocols.

The components of the IPM units 210A and 210B, including the wheels 220, 230, and 240, and the transmission screw 250 and the implant carrier 260, may be made of materials that are both biocompatible and compatible with a specific sterilization method, such as gamma or ethylene oxide. The components may be made of metal or plastic. Examples of the metals for fabricating the components may include stainless steel, cobalt chromium, or titanium, among others. Examples of plastics for fabricating the components may include Acrylonitrile-Butadiene-Styrene (ABS), Polycarbonate, Polyetheretherketone, or Polysulfone, among others.

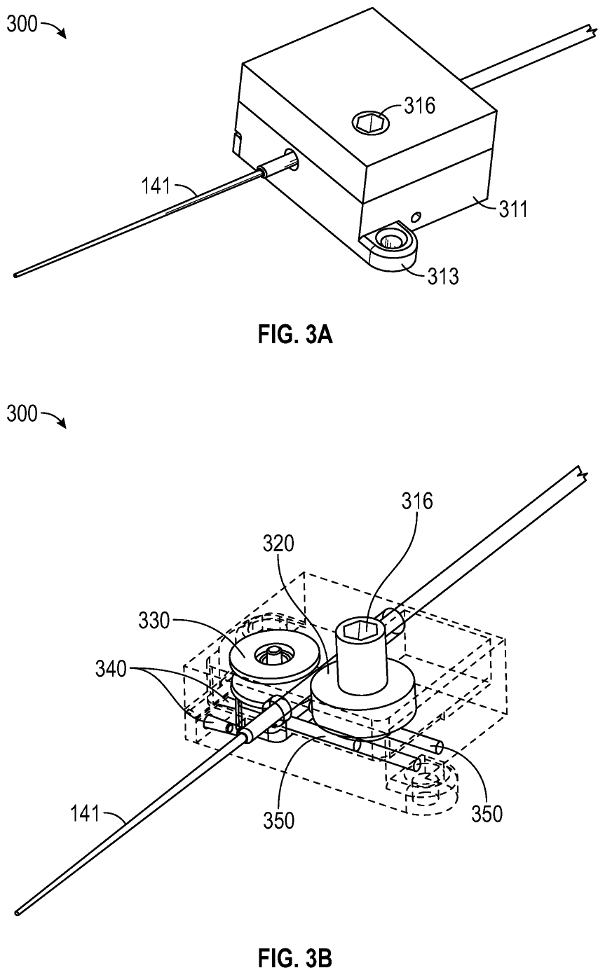

FIGS. 3A-3B are diagrams illustrating an example of an IPM unit 300, which is an embodiment of the IPM unit 210A as illustrated in FIG. 2A. The IPM unit 300 is configured to receive a user's direct control of a coupling unit that engages and moves the elongate member 141 of an implant. FIG. 3A illustrates an external view of the IPM unit 300, which includes a housing 311 to enclose a coupling unit and other components, one or more fixation flanges 313 for affixing the IPM unit 300 to a body part or a sterile field of surgery, and a recessed engagement unit 316 for engaging with the user control device. FIG. 3B illustrate internal components of the IPM unit 300, including a drive wheel 320, an idler wheel 330, and a slide set 350. The recessed engagement unit 316, which is an embodiment of the engagement unit 216, has specific geometry and size that match the engagement unit of the user control device. In the illustrated example, the recessed engagement unit 316 includes a hexagonal socket that matches an Allen key portion of the user control device. The recessed engagement unit 316, and the hexagonal socket, may be concentric with the drive wheel 340. In an example, the recessed engagement unit 316 may be part of the axle shaft of the drive wheel 340. Rotation of the recessed engagement unit 316, such as produced by the user control device, would drive rotation of the drive wheel 320.

The idler wheel 330, which is an embodiment of the idler wheel 230, may be laterally spring loaded using springs 340 connecting an axel shaft of the idler wheel 330 to the housing 311. The spring-loaded idler wheel 230 can accommodate and adjust to implants with elongate member of varying outside diameters. The spring-loaded idler wheel 330 may be attached to one or more slides 350 that is spring biased against the drive wheel 320 via compression springs. In an example, the one or more slides 350 include dowel pins situated in respective accommodating holes. The holes may be sized, shaped, or surface-processed to improve smooth sliding of the dowel pins under the spring tension with less friction. The elongate member 141 of the implant is advanced forward by rotating the active wheel 320 (such as activated by the user control device via the recessed engagement unit 316), which compresses against the idler wheel 330 and interfaces to the elongate member 141 via friction. In some examples, the interacting surface of the wheel can include a friction increasing texture to prevent slippage of the implant on the wheels.

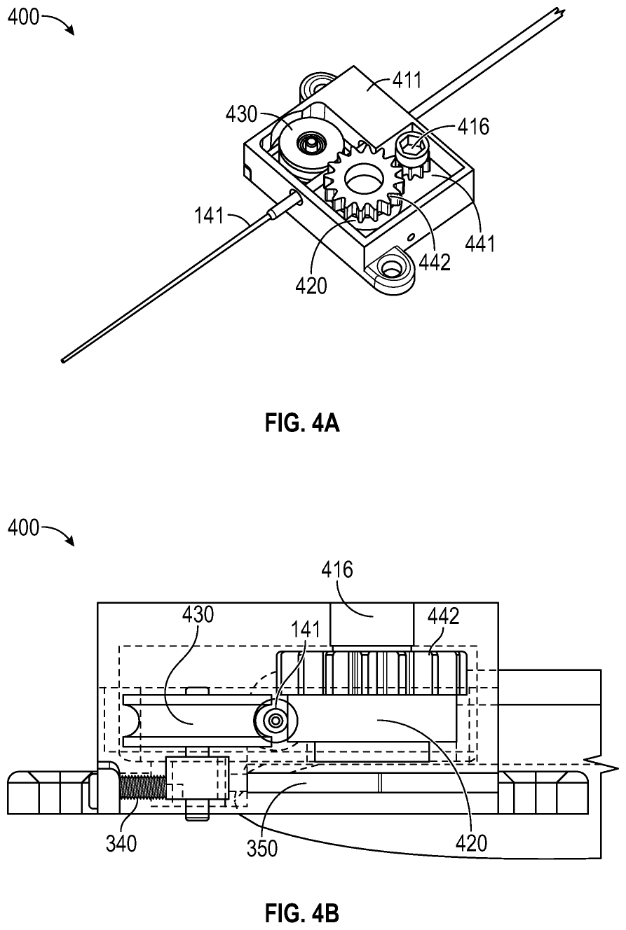

FIGS. 4A-4B are diagrams illustrating an example of an IPM unit 400, which is another embodiment of the IPM unit 210A as illustrated in FIG. 2A. FIG. 4A illustrates internal components, and FIG. 4B is a cutaway diagram illustrating interconnections among some of the internal components of the IPM unit 400. Similar to the IPM unit 300, the IPM unit 400 includes an engagement unit 416 to receive driving force from a user via a user control device, and a drive wheel 420 and a spring-loaded idler wheel 430 compressed against each other. Rotation of the wheels 420 and 430 can frictionally propel the elongate member 141 of the implant at specific direction and rate. The spring-loaded idler wheel 430 may be laterally spring loaded using springs 340 connecting an axel shaft of the idler wheel 430 to the housing 411, and can move along the slides 350 under the spring tension with less friction. Compared to IPM unit 300 which receives user direct control of the drive wheel 320, the IPM unit 400 allows a user to control the drive wheel 420 indirectly through a transmission unit. The transmission unit can include a gear set configured to transmit the force and motion from the engagement unit 416 to the drive wheel 420. In an example, the gear set can achieve a specific gear reduction. A user (e.g., a surgeon) can use the gear reduction to achieve more precise and fine-resolution control of motion and position of the implant. As illustrated in FIG. 4A-4B, the gear set 440 includes at least a first spur gear 441 coaxially coupled to the engagement unit 416 and a second spur gear 442 coaxially coupled to the drive wheel 420. The first spur gear 441 is a smaller gear with smaller number of teeth, which meshes with and drives the second gear 442 which is a larger gear with greater number of teeth. With a gear ratio greater than 1:1, the rotational speed of the second spur gear 442 is reduced by dividing it by the gear ratio. In some examples, the gear set may include one or more intermediate gears between the first and second gears.

FIGS. 5A-5B are diagrams illustrating an example of an IPM unit 500, which is another embodiment of the IPM unit 210A as illustrated in FIG. 2A. FIG. 5A illustrates an external view of the IPM unit 500. FIG. 5B illustrates a part of internal components of the IMP unit 500. The IPM unit 500 includes a recessed engagement unit 516 for engaging with the user control device. The recessed engagement unit 516, which is an embodiment of the engagement unit 216, includes a hexagonal socket to match an Allen key portion of the user control device. The recessed engagement unit 516, and the hexagonal socket, may be concentric with a worm 541. In an example, the recessed engagement unit 516 may be part of the axle shaft of the worm 541.

A user can indirectly control the drive wheel 520 through a transmission unit configured to transmit the force and motion to the drive wheel 520, and change the direction and/or speed of the motion applied to the recessed engagement unit 516. As an alternative to the spur gear arrangement in the IPM unit 400, the IPM unit 500 achieves gear reduction using a worm-gear arrangement, which involves the worm 541 meshing with a worm gear 542. The worm gear 542 may be coaxially coupled to the drive wheel 420. Large turns of the worm 541 can translate into small amount of rotation of the drive wheel 520, thus small distance that the implant travels. This would achieve a slow and steady insertion of implant via manual control, thereby increasing the resolution of implant motion control. The worm-gear arrangement is a compact means of achieving gear reduction and increasing torque. With less space requirement, the IPM unit 500 can have smaller physical size and lighter weight, which is advantageous for subcutaneous implantation. In some examples, additional gears or worms may be included and configured to achieve a desired gear reduction.

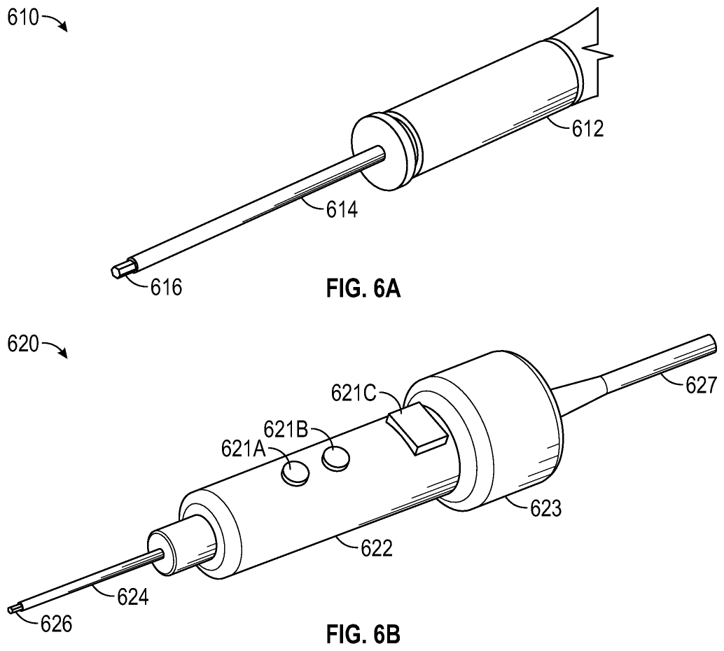

FIG. 6A-6B illustrate examples of user control devices that may be operatively coupled to the IPM unit 110 (or an embodiment thereof, such as the IPM units 210A, 210B, 300, 400, or 500) to activate the coupling unit 111, directly or indirectly, to move an implant. Depending on whether the IPM unit 110 is configured for external or subcutaneous fixation, the user control devices may be used for external or percutaneous engagement with the IPM unit 110. FIG. 6A illustrates a manually-controlled device 610, and FIG. 6B illustrates a powered device 620. Both the manual device 610 and the powered device 620 include respectively a handle 612 or 622, a shaft 614 or 624, and an engaging tip 616 or 626, as illustrated in FIGS. 6A-6B. The handles 612 and 622 may be sized, shaped, and surface-processed to improve grip by the user. The shafts 614 and 624, which connect the respective handles 616 and 626 and the respective engaging tips 616 and 626, may be made of metal (e.g., steel) to resist bending or twisting. In an example, the shafts 614 and 626 can have a shape of a needle or a thin, minimally invasive rod with distal engaging tips 616 and 626. The engaging tips 616 and 626 can have size and shape that match the engagement unit of the IPM unit. In an example, the engagement unit of the IPM unit can have a recessed portion, such as a hexagonal socket as illustrated in 316, 416, or 516 of various embodiments of IPM units as discussed above, and the engaging tips 616 and 626 can include an Allen key portion that matches the hexagonal socket.

In some examples, the engaging tips 616 or 626 may be an interchangeable tip held onto the respective shafts 614 or 624 mechanically or magnetically. Engaging tips of different sizes and geometry may be detachably coupled to the shaft, and operably fit to the engagement units on the IPM unit 110 having a matching size and geometry. Interchangeable tips may not only improve the usability and flexibility of the user control device, but also provides an easy and cost-saving solution of replacing an worn or damaged engaging tip instead of replacing the entire user control device.

To use the manual device 610, a user may hold and twist the handle 612 of the clockwise or counter-clockwise to apply the force and torque to the engagement unit of the IPM unit 110, in a similar fashion as screwing or unscrewing a screw. The powered device 620 as illustrated in FIG. 6B includes additional components configured to generate and apply motorized force and torque to the engagement unit of the IPM unit 110. The powered device 620 can include a motor compartment 623, optimally extended to the internal of the handle 622. The motor compartment 623 can house an electric motor and a transmission unit configured to generate and transmit the driving force to the engaging tip 626, and rotate the engaging tip 626 at desired speed, direction, and torque, etc. The power transmission unit may include one or more of chains, belts, gears, or shaft couplings, among others. In an example, the powered device 620 is a cordless device that includes a power source (e.g., a battery) disposed within the motor compartment 623. In another example, the powered device 620 may be powered by an external power source connected to the motor via a cord 627. In various examples, the motor and transmission unit may be at least partially located externally to the motor compartment 623. Motorized control of the IPM unit 110 via the powered device 620 can help increase consistency of implant motion and decreased variability, which may lead to less trauma during the procedure.

The powered device 620 may include a user interface configured to receive motion control instructions from a user. As illustrated in FIG. 6B, the user interface includes one or more control buttons 621A-621C for a user to adjust the movement of the elongate member 141. Examples of the motion control parameters may include a target movement rate, a target movement direction or orientation, a target movement distance, a target position of a distal end of the elongate member, or a target amount of force imposed on the elongate member 141, among others. In an example, the control buttons 621A and 621B may be used to control the rotation direction of the engaging tip 626. For example, the engaging tip 626 can rotate clockwise when the control button 621A is pushed and held, or rotate counter-clockwise when the control button 621B is pushed and held. Through the engagement unit on the IPM unit 110, said rotations of the engaging tip may respectively drive the implant to move forward or backward. The control button 621C may be used to set the rotating speed of the engaging tip 626. In an example, pushing the button 621C in distinct patterns can increase or decrease the present rotation speed. The speed may be adjusted in a step-wise fashion. Accordingly, the implant movement speed may be incrementally or decrementally adjusted. In an example of cochlear implant surgery, the target movement rate is approximately at 100-micron intervals. Motorized fine control of implant speed via the powered device 620 can minimize peri-surgical tissue trauma or damage to the implant.

It is to be understood that the control buttons 621A-621C are shown in FIG. 6B as push buttons, this is by way of example and not limitation. Various control devices with different size, shape, or mode of adjustment, such as a switch button, slide bar, trackball, may be used to adjust one or more motion parameters. In some examples, at least a portion of the user interface (e.g., control buttons 621A-621C) may be implemented in a control console (not shown) separate from, and electrically coupled to, the powered device 620 via the cord 627.

In various examples, the manual device 610 or the powered device 620 may receive information about the force and motion of the implant. Such information may be sensed by sensors disposed in the manual device 610 or the powered device 620, or associated with the IPM unit 110. The for and motion information may also be received from an external system. Additionally, electrophysiological measurements may also be received, such as intraoperative ECoG, cochlear microphonics (CM), auditory nerve neurophonics (ANN) that can reflect immediate changes in the cochlear mechanics and insertion trauma pre-, during-, and post-insertion of the electrode array.

The manual device 610 or the powered device 620 may include a display unit to display the motion and/or electrophysiological measurement. In an example, the button 621C may include a data readout window for displaying, among other information, the rotation speed of the engagement tip 626, or calibrated movement rate of the implant. This allows a surgeon to monitor in real time the implantation progress, and adjust the force, speed, torque, direction, or other motion control parameters as needed. In an example, a visual indicator, such as a light emitting diode (LED) or an on-screen indicator on the display screen, may be generated. A specific LED color or a specific blinking pattern may signal to the user a successful positioning of the implant at the target implantation site. A different LED color or a different blinking pattern may alert an excessive force imposed on the implant due to unintended tissue resistance during the implant advancement. In an example, an audio indicator may be generated to alert the user of current motion and/or electrophysiological measurement. The audio indicator may include a beep with a specific tone, a specific frequency, or a specific pattern (e.g., continuous, intermittent, pulsed, sweep-up, or sweep-down sound). In an example, a beep with a specific tone or pattern may signal to the user successful positioning of the implant at the target implantation site. A beep or an alarm with a different tone or different pattern may alert an excessive force imposed on the implant. In an example, the beep or the alarm may go off continuously as the sensor senses the implant approaching the target site. The sound frequency or the pulse rate of the beep or the alarm may increase as the implant gets closer and finally reaches the target site. In an example, the frequency of the beep or the alarm may correspond to a rate of motion, such as sounding for every one millimeter of motion. Audible feedback on the motion parameters may be advantageous in that the surgeon may be notified in real time the implantation status or events encountered without the need to look away from surgical field. This may assist the surgeon with enhanced surgical precision and patient safety. In some examples, the audible or visual sensor feedback may signal to the user that the sensed implant position, motion, or force has exceeded maximum parameter values. In some examples, the display unit and audio feedback generator may be implemented in the external control console electrically coupled to the manual device 610 or the powered device 620. The user may adjust the force and/or motion output according to such measurement data or in response to the alert notification.

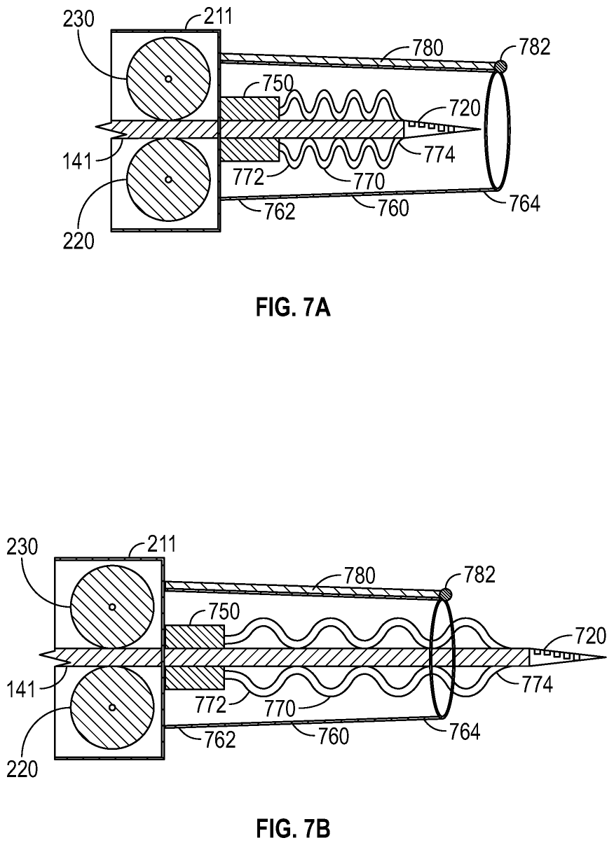

FIGS. 7A-7B are cutaway diagrams illustrating an example of portions of an IPM unit that includes a sheath 760 and optional sheath bellows 770, that enclose at least a portion of the elongate member 141 of the implant. The sheath can protect the elongate member 141 and the implant electrode array 720 from the surrounding environment. Fibrotic tissue can grow over the implant and often impede implant functionality and can significantly limit or prevent implant position changes or movement depending on the extent of tissue growth. With fibrotic tissue overgrowth, if a surgeon or clinician desires to remove, exchange, or adjust an implants position in the body, extensive dissection of healthy tissue and structure disruption may be necessary after the initial surgery or procedure, which may lead to increased surgical complication or high risk of inadvertent damage to healthy structures. The sheath 760 can prevent the impeding, fibrotic tissue overgrowth.

The sheath 760 has a proximal end 762 and a distal end 764. The proximal end 762 may be tapered from an initial larger diameter to a smaller diameter at the distal end 764. The proximal end 762 may be connected to a sheath anchor element 750 affixed to the housing 211 of the IPM unit for a hermetic or watertight seal. This may be achieved through direct fusion with the case material, screw, barb, clamp, or other means to seal the tubing to the case. The distal end 764 opens at a surgical entrance of the target implantation site (e.g., the entry into the cochlea). The elongate member 141 may be flexible and prone to twisting, entanglement, or buckling. The sheath 760 may at least partially enclose the elongate member 141 to provide resilient support to the elongate member 141 and the electrode array 720, thereby keeping the implant on track between the housing 211 and the surgical entrance of the target implantation site. It may also protect electronics such as an electrode array positioned on the elongate member 141 and the conductors inside the elongate member 141.

The sheath 760 may be composed of biocompatible materials, such as polymeric (polyimide, polytetrafluoroethylene (PTFE), silicon), thermoplastic, metals, or composite materials (non-ferromagnetic coil or braid reinforced) which may be permanently implanted. The sheath 760 may be flexible, rigid, semi-rigid, gas permeable or impermeable, fluid permeable, selectively permeable or a combination of varying physical and chemical properties tailored to the housed implant requirements. The sheath 760 comprises a flexible tube whose dimensions may substantially match the elongate member 141. For example, the diameter of the tube may be slightly greater than the diameter of the elongate member 141, such that the flexible tube may provide desired rigidity to the elongate member 141 and the electrode array 720 inside; while at the same time does not produce undue friction between the elongate member 141 and the interior surface of the tube. To decrease friction, the sheath 760 may be pre-lubricated with a biocompatible and sterilizable lubricant. Alternatively or additionally, the interior surface of the tube may be treated with functionalizing materials for decreased internal friction, such as PTFE, or include linear grooves, pattern, rings or guidance tracks. These internal luminal surface features have several functions such as decreasing inner surface friction, guide moving electrode/filament to maintain specified configurations (i.e. rotational or spatial relationship) or preventing passage of intraluminal fluids or materials.

In some example, the sheath 760 supports and internally houses an insertion tube in a telescoping fashion. The insertion tube can slide within the sheath 760 and over the electrode array 720 also housed within. The insertion tube moves through the sheath 760 (affixed to the IPM unit proximally) and over the electrode array 720 on the abluminal side within. This enables controlled movement of both the insertion tube and the electrode array 720 into the delicate intracochlear space.

The coupling units, such as the wheels 220 and 230, may frictionally drive the elongate member 141 to move inside the sheath 760, such as from the proximal end 762 toward the distal end 764 or from the distal end 764 to the proximal end 762 inside the sheath 760. The elongate member 141 and the electrode array 720 can travel smoothly inside the tube of the sheath 760, including forward, reverse, lateral, or rotational in multiple degrees of freedom yet limited by wall architecture of the sheath 760 or surrounding anatomical limitations.

The distal end 764 of the sheath may be fixed or reversibly stabilized at a designated position of the surgical opening of the implantation. In an example of cochlear implant, the distal end 764 may be stabilized at the cochlea round window (RW) or cochleostomy site by closely matching tube diameters to the RW niche or cochleostomy dimensions. This would allow the sheath 760 to be compressed into the RW niche, or temporarily through the RW membrane. In some examples, the sheath 760 may be detached from the implant once the implant is positioned at the target site of implantation.

The sheath 760 may be a fully implantable sheath. In some examples, the sheath 760 is a drug-eluting sheath that stores within its lumen, or coupled within its interior walls, drugs for the purpose of eluting various pharmacological agents such as antibacterial, antifouling, antiproliferative, or antifibrotic drugs. This can prevent tissue formation and fibrosis within the sheath 760 that would hinder implant movement. In some examples, the pharmacological agents may be stored in a drug reservoir within the housing 211. The drug reservoir is connected to the lumen of the sheath 760 through a conduit. The pharmacological agents may be released to the lumen of the sheath 760 at a controlled rate or at specific time intervals. In some examples, the sheath 760 may additionally be used to provide liquid delivery of therapeutic agent via a double lumen or a single lumen design.

In some examples, a physiologic sensing probe 780 may be attached to the exterior of the sheath 760. The sensing probe 780 may include a distal sensing electrode 782 that makes direct contact with the bone at the round window niche via a ball tip or wire loop end to sense a physiologic signal, such as such as ECoG, during cochlea implantation. The sensing probe 780 may include electrically conductive wires encompassed in the sheath material or via a separate sheath channel to conduct the sensed signal. The ECoG signal obtained at the round window via the sensing electrode 782 may be presented to a user via an output unit. The user may adjust the driving force to optimize implant positioning using the ECoG feedback.

The sheath bellows 770, in the form of flexible and expandable impermeable membrane, may be provided within the sheath 760. The sheath bellows 770 can seal the elongate member 141 but leave the electrode array 720 uncovered. The sheath bellows have a proximal end 772 attached to the sheath anchor element 750, and a distal end 774 attached to a distal portion of the elongate member 141 in proximity to the electrode array 720. FIG. 7A illustrates the sheath bellows 770 being compressed under pressure within the sheath 760 before the elongate member 141 and electrode array 720 and delivered and deployed. FIG. 7B illustrates during the implant delivery, as the elongate member 141 is moved forward (e.g., by the wheels 220 and 230) and the electrode array 720 advances to the implantation site, the pressure on the sheath bellows 770 is released. Accordingly, the sheath bellows 770 extends and moves forward along with the elongate member 141 and electrode array 720. When the elongate member 141 is partially housed within the sheath 720, the sheath bellows 770 allows the elongate member 141 to move external to the sheath 760 at a future point in time while preventing fibrotic issue ingrowth around the implant or into the protective sheath.

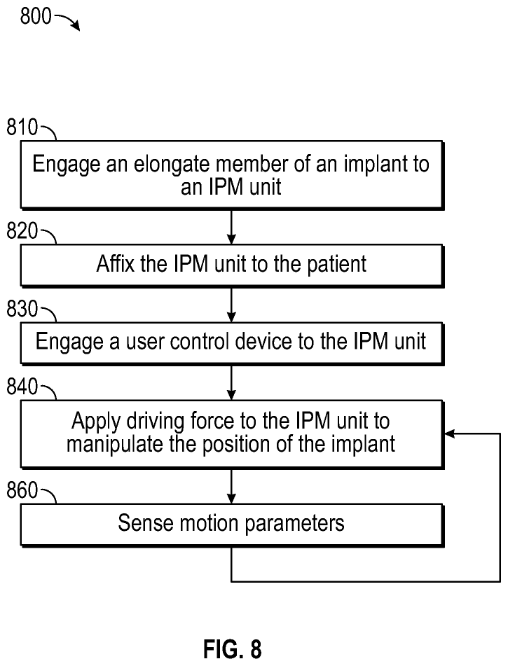

FIG. 8 is a flowchart illustrating, by way of example and not limitation, a method 800 for manipulating an implant's position in a patient via an external or subcutaneously implantable system such as the device-assisted implantation system 100. In an example, the method 800 may be used to operate an implant position manipulator (IPM) unit, such as the IPM unit 110 or any embodiments discussed above (e.g., IPM units 210A, 210B, 300, 400, or 500), to deliver and position a cochlear implant to a target cochlear region to treat hearing loss in a hearing-preservation cochlear implant surgery. The treatment may include electrostimulation, or other therapy modalities. In some examples, the method 800 may be used to operate the IPM unit to deliver, steer, position, or extract other types of implants or prosthesis. Examples of such implants may include leads, catheter, guidewire, sheath neuroprosthesis, implantable stimulator such as spinal cord stimulator, brain stimulator, or other tubular implanted medical devices. The implants may be used for diagnostic or therapeutic purposes.

The method 800 commences at step 810, where an elongate member of an implant (e.g., a cochlear implant) may be engaged to the IPM unit. In the case of implanting or adjusting the position of a cochlear implant for hearing-preservation surgery, the cochlear implant may include an implantable stimulator for subcutaneous implantation under the scalp. The implantable stimulator may generate electrostimulation impulses conducted to the electrode array for stimulating cochlear nerves. The cochlear implant may include an elongate member with an electrode array disposed at a distal portion of the elongate member. The IPM unit may include a coupling unit to interface with the elongate member. In an example, the coupling unit may comprise roller arrangement including at least a drive wheel and an idler wheel, as illustrated in FIGS. 2A, 3A-3B, 4A-4B, and 5A-5B. The elongate member of the implant may be fed through the IPM unit via an entrance port and an exit port, and pinched between the drive wheel and the idler wheel. The idler wheel may be spring-biased and compress against the drive wheel, via a torsion spring. The torsion spring may be manually biased to release the compression and open the space between the drive wheel and the idler wheel to accommodate the elongate member into the IPM unit. In another example, the coupling unit may comprise a roller-implant carrier arrangement, and a transmission screw can convert rotary motion of a drive wheel to linear motion of the implant carrier, thereby moving the elongate member of the implant forward or backward.

At 820, the IPM unit may be affixed to a patient, such as on the patient head to maintain sufficient stability during the advancement of the implant. The IPM unit may alternatively be securely attached to an object at the patient's immediate environment such as an equipment attached to a surgical table. The IPM unit may alternatively be subcutaneously implanted. As previously discussed with reference to FIG. 1, the IPM unit may be sized and shaped to facilitate patient attachment. The IPM unit may include a fixation member, such as one or more of a screw, a pin, a nail, a wire, a hook, a suture, or a magnet. The IPM unit may have an exterior contact surface with a rough texture, or equipped with one or more gripping elements. Examples of the gripping elements may include penetrators such as spikes, pins, or barbs protruding from the exterior surface. When the exterior contact surface is in contact with a body part of the patient (e.g., patient head) and the IPM unit is pressed and held against the body part, the gripping elements may provide sufficient friction or gripping force to securely hold the IPM unit in place during the implant advancement.

At 830, a user control device may be engaged with the IPM unit. This may be done manually by a user (e.g., a surgeon). The IPM unit may include an engagement unit directly or indirectly (e.g., via a transmission unit) coupled to the coupling unit (e.g., the drive wheel) of the IMP unit. The engagement unit may be positioned subcutaneously under the skin or other minimally invasive site and enables controlled movement of an implant deep to tissue, bone, or other delicate organ structures. The engagement unit may include physical, optical, or magnetic identifiers to allow a user to easily locate the access point in a future point in time when the implant needs to repositioned The user control device may be a mechanical device, such as device 610 as illustrated in FIG. 6A, which has a matching engagement unit sized and shaped to match the engagement unit on the IPM unit. In an example, the engagement unit of the IMP unit includes a hexagonal socket, and the engagement unit of the user control device includes an Allen key that matches the hexagonal socket. Alternatively, the user control device may be a powered device, such as device 620 as illustrated in FIG. 6B, which has a motor and a transmission to supply the force and motion to the IPM unit via the engagement unit. Alternatively, the user control device may engage with the IPM unit via magnetic or electromagnetic coupling.

At 840, driving force may be applied to the IPM unit, using the user control device, to manipulate the position of the implant. In an example, a user may manually apply the force, such as by using the manual device 610, to the engagement unit of the IPM unit. In another example, a user may operate a powered device 620, the motor therein generates the force and motion that drive the IPM unit via the engagement unit. In an example of cochlear implant, the electrode array of the cochlear implant may be inserted into and positioned at the target cochlear site. In patients with impaired high-frequency hearing function but preserved low-frequency hearing function, a short electrode array of the implant may be positioned at the outer or basal cochlea. Electrostimulation may be delivered therein via the electrode array to restore high-frequency hearing function.

In some examples, at least a portion of the elongate member of the implant may be situated in a sheath and/or sheath bellows, such as describe above with reference to FIGS. 7A-7B. The sheath and/or sheath bellows can prevent fibrotic tissue overgrowth over the implant, thus maintaining physical and electrical properties and proper functioning of the implant. In some examples, the sheath may internally house an insert tube in a telescoping fashion. In another example, the sheath bellows extends and moves along with the elongate member. The method at 850 may further including operations of delivering and/or positioning a sheath and/or sheath bellows and determining whether the sheath has reached a target positon. Monitoring the sheath and/or sheath bellows insertion can involve similar sensor feedback as used with implant insertion.

The method 800 may include an optional step 860 of sensing one or more motion parameters, such as via one or more sensors disposed in the user control device or within the IPM unit (e.g., at the drive wheel or idler wheel, or the implant carrier). Examples of the sensors may include linear or rotary encoders, ferromagnetic Hall effect sensors, optical sensors, capacitive sensors configured to detect implant motion and/or position, or force sensors configured to sense indications of force or friction imposed on the implant during the implant advancement, such as axial, lateral, or radial insertion force as the implant advances into the cochlea. The sensor information may be communicated to a receiver, such as implemented in the user control device, via a wired connection or a wireless network, such as near-field communication (NFC) protocols. The sensor information may be presented to a user, such as being displayed on a display unit on an external monitor, or on the user control device. In addition to the force and motion parameters, electrophysiological measurement may be recorded and presented to the user, which may include intraoperative ECoG, cochlear microphonics (CM), auditory nerve neurophonics (ANN), among others. The presentation may include real-time visual or audible notification with specified patterns corresponding to different types of events encountered during implantation. The audible and visual feedback may also signal to the user that the sensed implant position, motion, or the forces may have exceeded the target parameter values.

The sensor information may be checked to determine whether target implantation site has been reached, or certain events have occurred as a result of present implant manipulation. A target implantation site is reached if the sensed distance of insertion reaches the user programmed target distance within a specified margin. A user may adjust the force and/or motion output according to such measurement data or in response to the alert notification at 850. If it is determined that the target implantation site has been reached, then the implant may be released and positioned at the target implantation site, and the IPM unit may be removed.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

1. A system for positioning an implant in a patient, the system comprising:
an implant position manipulator (IPM) unit, including a coupling unit configured to reversibly engage an elongate member of the implant and to deliver and position the implant into a target implantation site; and
a user control device operably coupled to the IPM unit, the user control device configured to apply driving force to the IPM unit to control delivery and positioning of the implant into the target implantation site.

2. The system of claim 1, wherein the implant includes a cochlear implant having an electrode array disposed on a portion of the elongate member.

3. The system of claim 1, wherein the user control device is operatively coupled to the IPM unit via a mechanical coupling including an engagement unit of the user control device and an engagement unit of the IPM unit, wherein the engagement unit of the user control device matches in size and shape of the engagement unit of the IPM unit.

4. The system of claim 1, wherein the user control device is operatively coupled to the IPM unit via a magnetic or electromagnetic coupling between a magnetic or electromagnetic coupler of the user control device and an magnetic or electromagnetic coupler of the IPM unit.

5. (canceled)

6. The system of claim 1, wherein the user control device is an unpowered device including a handle for a user to directly apply the driving force.

7. The system of claim 1, wherein the user control device is a powered device including an electric motor configured to produce the driving force to control the IPM unit.

8. The system of claim 7, wherein the powered device includes control mechanisms to adjust the driving force or a motion of the user control device.

9. The system of claim 1, wherein the user control device includes an output unit configured to present one or more motion parameters of the implant.

10. The system of claim 1, comprising a sensor configured to sense one or more motion parameters of the implant.

11.-14. (canceled)

15. The system of claim 1, comprising a physiologic sensor configured to sense one or more electrophysiological signals.

16. The system of claim 1, wherein:
the coupling unit includes at least first and second rollers arranged and configured to engage, through compression between respective radial outer surfaces of the first and second rollers, a portion of the elongate member of the implant; and
the user control device is configured to apply the driving force to the first roller to cause rotation thereof that propels the implant through friction generated by the compression.

17. The system of claim 16, wherein the IPM unit further includes a transmission unit configured to transmit the driving force to the first roller.

18. The system of claim 17, wherein the transmission unit includes a gear-reduction device configured to alter motion rate or direction produced by the driving force.

19.-20. (canceled)

21. The system of claim 16, wherein:
the IPM unit includes at least one slide spring-biased against the first roller via a compression spring; and
the second roller is spring loaded and configured to be attached to the at least one slide, and to engage at least a portion of the elongate member of the implant through compression between the respective radial outer surfaces of the first and second rollers.

22. The system of claim 1, wherein:
the coupling unit includes a drive roller and an implant carrier, the implant carrier configured to detachably hold at least a portion of the elongate member of the implant; and
the user control device is configured to apply the driving force to the drive roller to cause rotation thereof that propels the implant carrier along with the elongate member.

23. The system of claim 22, wherein the implant carrier is coupled to a drive roller via a transmission screw, the transmission screw configured to convert a rotary motion of the drive roller to a linear motion of the implant carrier.

24.-25. (canceled)

26. The system of claim 1, comprising a housing to enclose the IPM unit, the housing including a sealable opening for the user control device to access the IPM unit.

27. The system of claim 26, wherein the housing includes a viewing window to display an indication of implant position.

28.-29. (canceled)

30. The system of claim 1, wherein the IPM unit further includes a fixation member configured to detachably affix the IPM unit to the patient.

31. The system of claim 1, wherein the IPM unit is configured for subcutaneous implantation, and the user control device is configured for transcutaneous coupling to the IPM unit.

32. The system of claim 1, further including a sheath extended from the IPM unit to a surgical entrance of the target implantation site, the sheath configured to at least partially enclose the elongate member.

33. The system of claim 32, further comprising sheath bellows within the sheath, the sheath bellows configured to extendably seal the elongate member as the implant advances into the target implantation site.

34. The system of claim 32, wherein the sheath is a drug-eluting sheath.

35. The system of claim 32, wherein the IPM unit includes a drug reservoir to store pharmacological agents controllably released to the sheath.

36. A method for delivering and positioning an implant with an elongate member into a target implantation site of a patient via a device-assisted implant positioning system, the method comprising:
engaging at least a portion of the elongate member of the implant to an implant position manipulator (IPM) unit of the implant positioning system;
affixing the IPM unit to the patient via a fixation member of the IPM unit;
engaging a user control device to the IPM unit; and
applying driving force to the IPM unit to control delivery and positioning of the implant into the target implantation site.

37. The method of claim 36, wherein the engagement of the elongate member includes engaging at least a portion of the elongate member of the implant through compression between respective radial outer surfaces of first and second rollers; and
wherein applying the driving force includes driving rotation of the first roller that propels the implant through friction generated by the compression between the radial outer surfaces of the first and second rollers.

38. The method of claim 36, wherein the engagement of the elongate member includes attaching at least a portion of the elongate member of the implant to an implant carrier coupled to a drive roller via a transmission screw; and
wherein applying the driving force includes driving rotation of the drive roller that propels the implant carrier along with the elongate member.

39. The method of claim 36, further comprising enclosing the at least a portion of the elongate member of the implant within a sheath or sheath bellows.

40. The method of claim 36, wherein applying the driving force is through an electric motor within, or connected to, the user control device.

41. The method of claim 36, further comprising:
sensing one or more motion parameters of the elongate member or electrophysiological signals during the implantation of the implant; and
controlling the IPM unit to propel the implant according to the sensed one or more motion parameters.
